(19) United States
(12) Patent Application Publication
(10) Pub. No.: US 2012/0219621 A1
(43) Pub. Date: Aug. 30, 2012

(54) Composition with Enhanced Thermogenic Activity and the Use Thereof in the Prevention and Treatment of Obesity

(75) Inventor: Mariana Gabriela Alvarez Favela, Torino (IT)

(73) Assignee: MEDESTEA BIOTECH S.P.A., Torino (IT)

(21) Appl. No.: 13/501,453

(22) PCT Filed: Oct. 11, 2010
(86) PCT No.: PCT/IB2010/054593

§ 371 Date: Apr. 11, 2012

(30) Foreign Application Priority Data
Oct. 12, 2009 (IT) TO2009A000777

(51) Int. Cl. A61K 9/20; A61K 31/105; A61K 31/353; A61K 31/122; A61K 38/44; A61K 36/28; A61K 36/258; A61K 36/84; A61K 36/16; A61K 36/87; A61K 36/73; A61K 9/68; A61P 3/00; A61P 3/06; A61P 3/10; A61P 9/12; A61K 31/165

(52) U.S. Cl. 424/464; 514/627; 514/707; 514/456; 424/94.1; 514/690; 424/94.4; 424/764; 424/728; 424/733; 424/752; 424/766; 424/765; 424/48

(57) ABSTRACT

A composition with enhanced thermogenic activity and the use thereof in the prevention and treatment of obesity

Pharmaceutical compositions of a dietary supplement or medical device, useful for the treatment and prevention of overweight and obesity and metabolic syndrome comprising:
a) menthol or a peppermint essential oil comprising menthol,
   in association with one or more components having a thermogenetic activity, selected from:
b1) capsaicin or a capsacinoid derived from Capsicum genus or a Capsicum extract containing capsaicin and/or capsacinoids;
b2) a sulphur containing active principle derived from the Allium genus or an Allium extract containing it;
b3) a catechin active substance derived from the genus Camellia or a Camellia extract containing it.

The menthol and mint essential oil have a potentiating action on the biological activities of the other aforementioned components.

The present invention relates to a composition containing active substances with thermogenic activity aimed at the prevention and treatment of excess weight and obesity, and also useful in the treatment of hypercholesterolemia, hypertriglyceridemia, diabetes, hypertension and metabolic syndrome.

### STATE OF THE ART

Obesity, but also simple excess body weight, is currently one of the most significant healthcare problems in socioeconomically developed countries. Indeed, these excesses have negative consequences, both from a medical viewpoint (resulting in increased incidence of heart attack, stroke, atherosclerosis and all the other chronic-degenerative pathologies troubling a large number of modern societies, such as hypercholesterolemia, hypertriglyceridemia, diabetes, hypertension and metabolic syndrome) and also the aesthetic viewpoint. Indeed, it should be remembered that excess body weight have an influence on behaviour and self-esteem, with negative psychological consequences. It is thus obvious that, besides constituting a gain for the community at large, slimming with the aim of losing excess weight is crucial for the psycho-physical wellbeing of each individual.

In accordance with standard pharmacology, treatment aimed at weight loss is essentially based on the use of so-called “anorectics”. As is well known, those belonging to the amphetamine category should be used with extreme caution, since they can significantly aggravate pre-existing cardiovascular and respiratory disorders.

Even in cases where the use of amphetamines is permitted, the use should be extremely limited over time. Indeed, such drugs can be responsible for side-effects of significant severity (insomnia, irritability, gastrointestinal disorders) but, in particular, addiction and dependency.

Another category of compounds to be avoided is the diuretics. In addition to being useful in the treatment of hypertension, these are only justified in the treatment of severe obesity (overweight in excess of 35-40%) where fluid retention is detected. In all other dietary regimen, the risks associated with taking diuretics (electrolyte deficiency) greatly outweigh the potential advantages (weight loss associated with increased fluid excretion).

Another inappropriate therapy is the inappropriate use of potent laxatives. By drastically reducing the absorption of nutrients, these have a partial slimming effect, but are the source of persistent intestinal inflammation and vitamin and/or nutrient deficiency. Finally, considering the risk associated with the onset of such phenomena as addiction and dependency, it is easily understood how such remedies cannot be considered valid alternatives.

However, it is appropriate to also dwell upon another type of consideration: the reduction in body weight should be associated with reduced fat mass and not specific reduction in body mass, which, involuntarily but inevitable also leads to reduced muscle mass.

Through a significant and appreciable reduction in caloric intake, establishing a balance of approx. 1200-1700 calories/die (depending on whether dealing with female or male subjects), it is possible to obtain a reduction of approx. 1 kg approximately every 8-10 days (depending on the individual involved, age, sex etc.). However, this reduction is as much associated with a loss of fat mass as it is with loss of lean muscle mass. Indeed, in an attempt to obtain the energy required, having exhausted its carbohydrate reserves (in the liver and muscle), the human body starts to use body fat, but then unfortunately also uses protein (the latter being contained in the lean mass).

Indeed, thanks to this protein reserve, and its precise biochemical transformation, the body obtains those sugars essential for brain survival that cannot be obtained from body fat, except in the case of prolonged fasting. Indeed, it may be stated that “due to the brain” an overly drastic reduction in daily caloric intake (such as the wrong dietary regimen or harmful weight-loss therapy) leads to a reduction in body weight that also involves muscle mass. This is not advantageous. Indeed, it is always important for the body to preserve its muscle tissue. Indeed, besides any aesthetic considerations, lean mass is essential for body health, since basal metabolism is directly proportional to its residual mass.

The above points demonstrate that proper weight loss should be obtained by associating a balanced dietary intake with therapy based on those active substances that can effectively: specifically reduce the amount of adipose tissue present; specifically inhibit the formation of new adipose tissue accumulation; exercise a thermogenic action; speed up basal metabolism by modifying the respiratory quotient.

Recently, several pharmacological studies have highlighted how certain compounds, appropriately formulated, induce thermogenic (destruction of fat), apoptotic (programmed cell death) and/or preadipocyte dedifferentiation (the preadipocyte can “deflate”) phenomena, that can be “exploited” for the treatment of obesity. Compounds of this type, all of dietary origin, are essentially the capsacinoids (derived from Capsicum annuum; chilli pepper), ajoene (derived from Allium sativum, garlic), EGCG (derived from Camellia sinensis, unfermented tea).

However, with the exception of the unfermented tea derivatives, for which several authors have claimed anti-obesity and anti-cholesterol activities from 2000 onwards, the evidence proposing thermogenic properties for capsacinoids and garlic derivatives is much more recent, and until a couple of years ago, the only pharmacological evidence was that related to the “anti-arthritis/anti-arthrosis” activities of capsicum derivatives (applied topically) and “hypocholesterolemic/hypotensive” activities of garlic derivatives.

Capsaicin and capsaicinoids are very stable alkaloids: they remain unaltered for long periods of time, even after cooking and freezing. As with all capsaicinoids, capsaicin is an irritant and produces a burning sensation in contact with the mucosa, where it passes into solution and stimulates VR1 receptors (vanilloid receptor type-1), which in turn activate the protein VRL-1 (vanilloid receptor-like 1), which, under normal conditions, is activated at temperatures of between 43° C. and 52° C. Despite capsaicin being something of an irritant if taken orally, if administered topically, and at very low dose, it is effective at reducing pain, thanks to its localised analgesic activity, but has no effect on the inflammatory component of the disorder.

However, numerous controlled studies, conducted both in experimental models (genetically obese mice and rats) and under clinical conditions, have recently shown capsacinoids to be capable of reducing adipose tissue mass by 10-20% in rats with a reduction in body weight of approx. 2-2.5% over a monthly basis in obese subjects.

Belonging to the Liliaceae family, the bulbs of garlic, Allium sativum, are of interest, from a pharmacological and clinical viewpoint. Indeed, following extraction, a number of active substances can be concentrated, all sulphurated compounds, such as allicin, diallyl-sulphide, S-allylcystein, Y-glutamyl-cystein, allin and particularly, ajoene (monomer of allin). Overall, these compounds have obvious clinical activities: hypocholesterolemic, anti-platelet aggregation, fibrinolytic, anti-hypertensive and antiseptic (at a dilution of 1/100,000, allicin is active against a number gram-positive and gram-negative bacteria: staphylococcus, streptococcus, pneumococcus, intestinal bacteria).

Hypolipidemic and antiatherogenic activities have also been demonstrated in several studies. For example, derivatized garlic bulb extracts reduce hyperlipidemia and inhibit the formation of atherosclerotic plaques induced experimentally in rabbits by means of an atherogenic (hypercholesterolemic) diet.

Again in this case, it has only recently been discovered that garlic derivatives also have the ability to potentiate the effects obtained on lean mass by high dose administration of CLA (no less than 600 mg/kg/die for 120 days) in genetically obese animals.

On the other hand, as described above, for several years, it has been observed that in humans, the use of catechin derivatives, especially in gallate form, obtained by the extraction of the aerial unfermented parts of Camellia sinensis (green tea) results in a 4% increase in basal energy expenditure as a result of the daily administration of extract containing at least 270 mg of epigallocatechin gallate (EGCG). To date, many authors have investigated this thermogenic phenomenon, claiming an apparent clinical effect in weight reduction. The active constituents that may be obtained from the unfermented leaves of Camellia sinensis correspond to polyphenolic structures, belonging to the flavanol class.

Easily identifiable using HPLC-MS methods, they correspond chemically to epigallocatechin, catechin, epicatechin, epigallocatechin-3-O-gallate, gallocatechin-3-O -gallate, epigallo-3-O-methyl gallate and epicatechin-3-O-gallate. For reasons of simplicity, these fractions are frequently commonly referred to as “green tea catechins” Of the seven fractions mentioned above, undoubtedly, the most interesting, from the pharmacological viewpoint, is EGCG, so much so that very often, the entire activity of the product is related to the content of this active substance.

The bibliography reports bibliographic references concerning the activities of green tea, ajoene and capsaicinoids in the treatment of obesity.

### SUBJECT OF THE INVENTION

The present invention is based on the recognition of the fact that, surprisingly, a) menthol, or the mint essential oils containing it, significantly potentiate the pharmacological and clinical activities of b1) capsaicin or capsacinoids or extracts of the capsicum genus containing them, b2) the sulphurated active substances derived from the genus Allium or extracts of Allium containing them and b3) the catechin-derived active substances derived from the genus Camellia or extracts of Camellia containing them, both individually and in association with one another, wherein said potentiation is displayed in terms of both anti-obesity action, and in terms of duration (brevity) of treatment necessary to obtain the expected result.

Hence, a composition of interest for pharmaceutical use, or as a dietary supplement or as a medical device, having the characteristics defined in the following claims, constitutes a subject of the invention.

The use of a composition, as defined previously, for the preparation of a medical product, a dietary supplement or a medical device useful for the therapeutic and/or cosmetic treatment and the prevention of obesity and/or overweight conditions, in addition to the treatment and prevention of metabolic syndrome, hypercholesterolemia, hypertriglyceridemia, diabetes and hypertension, constitutes an additional subject of the invention.

The synergic action resulting from the use of mint essential oil in potentiating the activities of the aforementioned components is completely unexpected. Rather, the anti-IBS (Irritable Bowel Syndrome) action possessed by mint essential oil, used at 100% purity grade, and characterised by a value of no less than 50% menthol, has been known for several years. Indeed, to date, no less than sixteen clinical studies (double blind, placebo-controlled) have been published describing the use of mint essential oil in the treatment of irritable bowel syndrome and dyspepsia.

Obviously, these studies have involved thousands of adult patients. In some of these studies, the action of the product in IBS, affecting infants, has also been evaluated. All these studies have highlighted the strong validity of the product, which has been effective in reducing irritation and pain (with or without diarrhoea, with resolution of the latter) in between 73 and 95% of cases, even after just a few weeks of treatment.

The mechanism of action of menthol, and the essential oil containing it, is continually under investigation. To date, it appears certain that they act at the level of intestinal cell calcium (Ca⁺⁺) channels. In fact, antagonism of calcium channels may explain the relaxation action on smooth muscle, which can be demonstrated clinically in subjects treated with mint essential oil.

Blocking the calcium pump might block intestinal contraction, causing decontraction and the restoration of more rhythmic, non-spasmodic contraction. However, calcium channel antagonism does not exclude other potential mechanisms of action, which might in some way contribute to the anti-IBS efficacy of the product.

The anti-obesity action is thus greatly unexpected, and to date there is no possible explanation in terms of mechanism of action.

Within the scope of the present invention, mint essential oil is understood as being all the essential oils that may be obtained from the genus Mentha, preferably Mentha piperita, galenically formulated in preparations for uncontrolled release, or formulated to obtain time-dependent and/or pH-dependent controlled release (protected or colon-specific forms). Within the scope of the invention, the use of mint essential oil with a menthol titre greater than 30-35% is preferable; however, the use of essential oils with menthol titre in excess of 50%, up to the use of essentially pure menthol, is also considered.

Typically, mint essential oils for pharmaceutical use, that may be used within the scope of the invention include:

| | |
|---|---|
| menthol | 35-45% by weight |
| menthone | 15-32% by weight |
| menthyl acetate | 4-10% by weight |

inferior quantities of menthofuran, isomenthone, pulegone, neomenthol, piperitone and cineol.

In the compositions according to the invention, mint essential oil is typically used in quantities of between 0.1 and 1000 mg/dose/die, preferably between 1 and 100 mg/dose/die.

Within the scope of the invention, the capsacinoids that may be used are natural alkaloids present in the oleoresin extracted from the fruit of plants of the genus Capsicum; the use of natural or synthesised capsacinoids is included within the scope of the invention, for example, the use of capsaicin, hydrocapsaicin, homodihydrocapsaicin, nordihydrocapsaicin, homocapsaicin, synthetic capsainoids such as vanillinamide of nonanoic acid and mixtures thereof, are considered within the scope of the invention.

Use of a natural extract from the genus Capsicum is preferred. Typically, said extract is titred in capsaicinoids, with a titre of between 1 and 10%.

Extracts from the genus Capsicum are available commercially.

Said capsacinoids, which may be used individually or in mixtures with one another, even at levels of purity of 100%, are used in the compositions according to the invention, with oral capsacinoid dosages comprised of between 0.01 mg/dose/die and 1000 mg/dose/die, preferably between 0.1 and 10 mg/dose/die. The same dosages apply to the aforementioned extracts from the genus Capsicum.

As indicated above, the sulphurated active substances derived from the genus Allium include ajoene, allin, allicin, diallyl-sulphide, S-allylcystein and Y-glutamyl-cystein, and mixtures thereof. The use of commercially available extracts of Allium sativum, with an ajoene titre of between 0.1-10%, is preferred. With regard to the active substances from the genus Allium, and including the aforementioned extracts, in the composition according to the invention, dosages comprised of between 0.1 and 1000 mg/dose/die of the aforementioned sulphurated compounds, preferably between 1 and 100 mg/dose/die, are considered.

In particular, the catechin active substances derived from the genus Camellia include epigallocatechin, catechin, epicatechin, epigallocatechin-3-O-gallate, gallocatechin-3-O-gallate, epigallo-3-O-methyl gallate and epicatechin-3-O-gallate; said active substances may be used individually or in combination with one another; the use of epigallocatechin gallate or extracts from the genus Camellia, in particular Camellia sinensis, containing it, are preferred. Said extracts, which are available commercially, typically contain an epigallocatechin gallate titre comprised of between 20 and 99%.

In the compositions according to the invention, for the active substances from the genus Camellia, dosages of between 0.1 and 3000 mg/dose/die of catechin derivatives, preferably between 20 and 500 mg/dose/die, are considered.

As previously indicated, the scope of the invention includes the use of all possible combinations between mint essential oil and menthol and each of the three components b1), b2) and b3), considered both individually, and in any combination of any two of the three, and all three together.

In addition to the previously described associations, the compositions according to the invention may also include additional components and/or ingredients, potentially suitable for optimising clinical efficacy. In particular, selected from one or more of the following:

antioxidants, such as vitamin A, C and E; SOD; CoQ10; GSH; cysteine;

compounds and extracts derived from vines, pomegranate, blueberry, G. biloba, and silymarin;

soothing agents/hypnotics, antidepressants, adaptogens, especially if of a natural origin, in particular DMG, betaine, meteonine donors, Kawa-kawa, compounds derived from Hypericus, Valerian, Passiflora, Camomile, Escolthia, Rhodiola, Ginseng, Euleterococcus;

NO donors, in particular L-arginine, L-citrulline, L-ornithine;

substances acting on energy and/or fat metabolism, such as carnitine and polycosanols.

In accordance with standard biochemical and galenic principles, all derived associations may be formulated as simple tablets, film-coated tablets, multilayered and programmed release tablets, gastroprotected and colon-specific tablets; sublingual tablets; effervescent tablets; capsules; water-soluble and orosoluble sachets; chewable lozenges; sprays for oral use.

All associations may also be formulated for topical use in the form of medicated gels, creams, ointments, soaps and detergents. In this case, it is expected each active substance shall be used at between 0.001 and 10%.

Experimental Tests

The activity of mint essential oil and menthol in potentiating the pharmacological action of the aforementioned components and associations thereof, has been verified by means of experimental testing, the results of which are reported in tables 1 and 2 below.

Table 1 reports the values observed concerning the reduction in weight increase in g (M±SD) after 90 days with thyme essential oil (X mg/kg/die) and mint oil (5 mg/kg/die) in Ob gene knock-out mice.

TABLE 1

| | | weight | Weight | |
|---|---|---|---|---|
| GROUPS | No. | (t = 0) | (t = 90) | t₉₀/t₀ % |
| 1. Untreated | 12 | 650 ± 85 | 818 ± 102 | +25.8% |
| 2. Thyme oil | 12 | 628 ± 79 | 779 ± 82 | +24.0% |
| 3. Mint oil | 12 | 632 ± 81 | 733 ± 85* | +16.0% |

*p < 0.05 with respect to thyme oil and untreated

Table 2 reports the data observed concerning weight loss in kg at 90 days, observed in overweight subjects [BMI between 27 and 30] subjected to a hypocaloric (1800 kcal/die) and normal protein diet, treated with purified extracts of Capsicum [6 mg/dose/die] Allium,[10 mg/dose/die] and Camellia [300 mg/dose/die] with or without the addition of Mint essential oil at 5 mg/dose/die.

TABLE 2

| | | Weight | Weight | | |
|---|---|---|---|---|---|
| GROUPS | No. | (t = 0) | (t = 90) | Δ kg | Δ % |
| 1. Untreated group | 10 | 95 ± 11 | 92 ± 10 | 3 | 3.15 |
| (diet alone) | | | | | |
| 2. Capsicum | 8 | 97 ± 10 | 92 ± 9° | 5 | 5.15 |
| 3. Mint/Capsicum | 9 | 98 ± 12 | 89 ± 10* | 9 | 9.20 |
| 4. Capsicum + Allium + | 7 | 94 ± 12 | 84 ± 11° | 10 | 10.6 |
| Camellia | | | | | |
| 5. Mint + Capsicum + | 9 | 96 ± 13 | 80 ± 8* | 16 | 16.6 |
| Allium + Camellia | | | | | |

•Drop outs:
2 in group 2
3 in group 4
1 in groups 3 and 5
°p < 0.01 vs untreated
*p < 0.05 vs corresponding control without mint

From the data obtained, it may be observed that the use of mint essential oil in genetically obese mice reduces the increase in adipose tissue by 33% with respect to the controls, while the group treated with thyme essential oil showed no reduction in body weight increase. Controls performed using other essential oils, such as camomile, valerian and mallow oil have given results similar to those observed using thyme oil as control.

From the data reported in Table 2, it may be observed over a monthly basis that, in humans, the association of mint essential oil with extracts from Capsicum, or with purified capsaicinoids, increases weight loss by almost 1.4% compared to that obtained with Capsicum or titred capsacinoids alone, calculated over the initial body weight of overweight subjects.

Analogous results have been obtained by associating mint essential oil with derivatives extracted from garlic bulb or purified ajoene.

Furthermore, over a monthly basis, an increase in weight loss of 2%, calculated over initial weight, has been observed by measuring the weight loss obtained, by associating mint essential oil (5 mg/die) with a mixture consisting of catechins derived from green tea (used in a high oral bioavailability form, “phytosomes”, in quantities of 300 mg/die), Capsicum extract (6 mg/die) and Allium extracts (10 mg/die).

Again, using a gastroprotected oral formulation containing mint essential oil derivative (5 mg/die), capsaicinoids (6 mg/die), phytosomal green tea extract (300 mg/die) and garlic extract derivative (10 mg/die), preliminary testing has been conducted in patients diagnosed with metabolic syndrome, characterised by concomitant altered fat, carbohydrate, blood pressure parameters, and clearly overweight.

Indeed, metabolic syndrome (also known as pluri-metabolic syndrome, X syndrome and insulin-resistance syndrome), corresponds to a collection of several metabolic and non-metabolic cardiovascular risk factors, combined with the presence of evident insulin-resistance. Obviously, the presence of metabolic syndrome increases the risk of cardiovascular mortality, in a manner proportional to the number of risk factors present. The first definition of metabolic syndrome was proposed by the WHO in 1998, establishing the following criteria, still considered valid: insulin-resistance and/or diabetes plus at least 2 of the following conditions: hypertriglyceridemia (≧150 mg/dL), hypertension (greater than 160/90 mmHg), reduced HDL cholesterol (≦35 in men and 39 in women), central obesity (waist/hip ratio ≧0.90 in men and 0.85 in women), microalbuminuria (UAE≧20 mg/min).

Obviously, the therapeutic approach to the problem is multifactorial. Hence, drugs commonly used for hypercholesterolemia (statins), diabetes (metformin and analogues), hypertension (diuretics, ACE inhibitors and others) etc. are used. Indeed, to date, there is no therapeutic approach based on a single category of drugs.

For ethical reasons, testing has been conducted in patients already receiving treatment with statins (20 mg/die), metformin (500 mg/die) and furosemide (40 mg/die), and therapy has then not been interrupted. The enrolled patients (20, males) have then been randomized into 2 groups of 10. In addition to the ethical therapy described above, depending on the group, patients have been administered the formulation forming the subject of the study, or an analogous preparation in terms of shape and colour, but without active substances (placebo). The established endpoints have been evaluated at 90 days. The results are reported in the following tables. From these, it may be observed that the formulation has reduced glycosylated hemoglobin by 1% and basal glycemia in such an evident manner as to return it to almost normal conditions (table 3).

The lipid parameters are even more obvious (table 4), where the formulation has restored normal triglyceridemia and cholesterolemia levels. The same applies to diastolic and systolic blood pressure parameters (table 5). Finally, in treated subjects, body weight and BMI parameters have dropped by 11 and 4 points in absolute value (table 6). It should also be observed that the HDL value, not reported in the tables, has been normalised in 85% of patients, dropping below 35 mg/dL.

It appears likewise obvious that the reduced values sometimes observed for the placebo group (compared to the value of the parameter at t=0) are due to the fact that these patients have continued with the ethical therapy and have thus been subject to thorough dietary control (hypocaloric/normal protein diet of 1800 kcal/die).

From the points observed, it appears evident that the formulation in question is capable of counteracting the complex and multi-factorial conditions associated with metabolic syndrome in addition to “simpler” situations where the subject has only 1, and not all, of the changes described (hypercholesterolemia with or without hypertriglyceridemia, hyperglycemia, hypertension).

TABLE 3
changes in carbohydrate parameters in response to treatment in
patients (n = 10/group) with diagnosis of metabolic syndrome

| | % glycosylated | % glycosylated | Basal | Basal |
|---|---|---|---|---|
| | hemoglobin | hemoglobin | glycemia | glycemia |
| Treatment | t = 0 | t = 90 | t = 0 | t = 90 |
| Formulation | 7.9 ± 0.5 | 6.9 ± 0.7* | 157 ± 16 | 117 ± 19° |
| Placebo | 7.5 ± 0.7 | 7.3 ± 0.6 | 143 ± 12 | 149 ± 21 |

*p < 0.01;
°p < 0.05 (vs t = 0)

TABLE 4
changes in lipid parameters in response to treatment in patients
(n = 10/group) with diagnosis of metabolic syndrome

| | | | Total | Total |
|---|---|---|---|---|
| | Triglycerides | Triglycerides | cholesterol | cholesterol |
| Treatment | t = 0 | t = 90 | t = 0 | t = 90 |
| Formulation | 245 ± 40 | 175 ± 22* | 242 ± 25 | 182 ± 17* |
| Placebo | 257 ± 48 | 235 ± 29 | 235 ± 20 | 229 ± 19 |

*p < 0.01;
°p < 0.05 (vs t = 0)

TABLE 5
changes in blood pressure parameters in response to treatment
in patients (n = 10/group) with diagnosis of metabolic syndrome

| | Minimum | Minimum | Maximum | Maximum |
|---|---|---|---|---|
| | pressure | pressure | pressure | pressure |
| Treatment | t = 0 | t = 90 | t = 0 | t = 90 |
| Formulation | 97 ± 11 | 81 ± 9° | 163 ± 17 | 128 ± 19* |
| Placebo | 94 ± 13 | 92 ± 11 | 158 ± 20 | 149 ± 15 |

*p < 0.01;
°p < 0.05 (vs t = 0)

TABLE 6
changes in body weight and BMI parameters in response to treatment
in patients (n = 10/group) with diagnosis of metabolic syndrome

| | Weight | Weight | BMI | BMI |
|---|---|---|---|---|
| Treatment | t = 0 | t = 90 | t = 0 | t = 90 |
| Formulation | 93.5 ± 7.2 | 82.6 ± 5.4* | 29.0 ± 2.5 | 25.2 ± 3.0° |
| Placebo | 91.9 ± 6.7 | 88.3 ± 6.2 | 28.7 ± 2.9 | 28.2 ± 2.8 |

*p < 0.01;
°p < 0.05 (vs t = 0)

Finally, it should be noted that analogous results have been obtained enrolling female subjects (n=8). In these patients, also with metabolic syndrome, compared to placebo, it has been noted a 90 day reduction in cholesterolemia (25%), triglyceridemia (33%), basal glycemia (18%), glycosylated hemoglobin (mean of 1.2 percentage points), body weight (8%) and BMI (3 points, in absolute value).

### FORMULATIONAL EXAMPLES

In the formulational examples reported below, the terms Capsicum, Allium and Camellia shall be used, intending all the potential extraction forms thereof, even including the use of pure derivative, in this case, capsaicin, ajoene and EGCG respectively; in the formulational examples reported, the numerical value reported for each ingredient is indicative of the dosage in mg/dose/die).

| 1) Film-coated tablets | |
|---|---|
| Mint essential oil: | 5 |
| Capsicum extract: | 6 |
| Allium extract: | 10 |
| Camellia extract: | 300 |
| Microcel | 200 |
| Dicafos | 200 |
| Plant magnesium stearate | 40 |
| PVP CL | 40 |
| Silicon dioxide | 20 |
| Shellac | 50 |
| 2) Film-coated tablets | |
| Mint essential oil: | 10 |
| Capsicum extract: | 6 |
| Allium extract: | 10 |
| Camellia extract: | 300 |
| Carnitine | 200 |
| Microcel | 200 |
| Dicafos | 200 |
| Plant magnesium stearate | 40 |
| PVP CL | 40 |
| Silicon dioxide | 20 |
| Shellac | 50 |
| 3) “0”-shaped capsules | |
| Mint essential oil: | 5 |
| Capsicum extract: | 6 |
| Allium extract: | 10 |
| Camellia extract: | 300 |
| Microcel 101 | 50 |
| 4) Sachets | |
| Mint essential oil: | 5 |
| Capsicum extract: | 6 |
| Allium extract: | 10 |
| Camellia extract: | 300 |
| Fructose | 1000 |
| Methocel E5 | 20 |
| Aerosol | 50 |
| Acesulfame K sweetener | 10 |
| E110 | 2 |
| Citrus flavouring | 150 |
| 5) sachets | |
| Mint essential oil: | 5 |
| Capsicum extract: | 6 |
| Allium extract: | 10 |
| Camellia extract: | 300 |
| Sucrose | 2265 |
| Citric acid | 50 |
| Silicon dioxide | 20 |
| Flavouring | 150 |
| Acesulfame K | 15 |
| 6) dual-layer programmed release tablets | |
| NORMAL-RELEASE LAYER | |
| Allium extract: | 10 |
| Camellia extract: | 100 |
| Dicaphos | 304 |
| Aerosil | 3 |
| Plant magnesium stearate | 6 |
| Colourant | 1 |
| SLOW-RELEASE LAYER | |
| Mint essential oil: | 5 |
| Capsicum extract: | 6 |
| Metholose | 80 |
| Aerosil | 2 |
| Plant magnesium stearate | 3 |
| Microcel | 80 |
| Dicaphos | 164 |
| 7) oro-soluble formulation | |
| Mint essential oil: | 1 |
| Capsicum extract: | 3 |
| Allium extract: | 10 |
| Camellia extract: | 150 |
| Sorbitol | 160 |
| Orange flavouring | 20 |
| Mandarin flavouring | 5 |
| Acesulfame k | 2 |
| Aerosil | 5 |
| Fructose | 1566 |
| 8) chewable lozenge | |
| Mint essential oil: | 5 |
| Capsicum extract: | 6 |
| Allium extract: | 5 |
| Camellia extract: | 100 |
| Gum base | 800 |
| Aspartame | 2 |
| Acesulfame | 1 |
| Levilite | 20 |
| Talc O.P. | 20 |
| Plant magnesium stearate | 18 |
| Shellac | 12 |
| Xylitol | 250 |
| Gum arabic | 6 |
| Titanium dioxide | 6 |
| Carnauba wax | 0.2 |

### BIBLIOGRAPHY

Green Tea:

Effects of Catechin Enriched Green Tea on Body Composition
Wang H, Wen Y, Du Y, Yan X, Guo H, Rycroft J A, Boon N, Kovacs E M, Mela D J.
Obesity (Silver Spring). Aug. 13, 2009.

Greenselect Phytosome as an Adjunct to a Low-Calorie Diet for Treatment of Obesity: a Clinical Trial
Di Pierro F, Menghi A B, Barreca A, Lucarelli M, Calandrelli A. Ahern. Med. Rev. June 2009; 14(2):154-60.

Ajoene:

Phytochemicals and Regulation of the Adipocyte Life Cycle
Rayalam S, Della-Fera M A, Baile C A.
J. Nutr. Biochem. Nov. 19, 2008 (11):717-26.

Molecular Mechanisms of Apoptosis Induced by Ajoene in 3T3-L1 Adipocytes
Yang J Y, Della-Fera M A, Nelson-Dooley C, Baile C A.
Obesity (Silver Spring). Mar. 14, 2006 (3):388-97.

Novel Treatments for Obesity and Osteoporosis: Targeting Apoptotic Pathways in Adipocytes
Nelson-Dooley C, Della-Fera M A, Hamrick M, Baile C A.
Curr. Med. Chem. 2005; 12(19):2215-25.

Capsacinoids:

Effects of Novel Capsinoid Treatment on Fatness and Energy Metabolism in Humans: Possible Pharmacogenetic Implications
Snitker S, Fujishima Y, Shen H, Ott S, Pi-Sunyer X, Furuhata Y, Sato H, Takahashi M.
Am. J. Clin. Nutr. January 2009; 89(1):45-50.

Capsaicin, a Spicy Component of Hot Peppers, Modulates Adipokine Gene Expression and Protein Release from Obese-Mouse Adipose Tissues and Isolated Adipocytes, and Suppresses the Inflammatory Responses of Adipose Tissue Macrophages
Kang J H, Kim C S, Han I S, Kawada T, Yu R.
FEBS Lett. Sep. 18, 2007; 581(23):4389-96.

1-12. (canceled)

13. A composition comprising:
a) menthol or a peppermint essential oil comprising menthol,
in association with one or more components having a thermogenetic activity, selected from:
b1) capsaicin or a capsacinoid derived from Capsicum genus or a Capsicum extract containing capsaicin and/or capsacinoids;
b2) a sulphur containing active principle derived from the Allium genus or an Allium extract containing it;
b3) a catechin active principle derived from the genus Camellia, or a Camellia extract containing it, for use in the treatment and prevention of obesity, overweight, metabolic syndrome, hypercholesterolemia, hypertriglyceridemia, diabetes or hypertension.

14. A composition according to claim 13, wherein independently one from the other:
component a) is an essential oil of Mentha piperita;
component b1) is a purified capsacinoid, capsaicin included, or a Capsicum extract;
component b2) is an Allium extract containing ajoene;
component b3) is a Camellia extract (green tea).

15. A composition according to claim 14, wherein the Camellia extract is present as a complex with soya distearoyl phosphatidylcholine.

16. A composition according to claim 13, comprising the following amounts of components expressed as mg/dosage unit/die:
a) peppermint essential oil from 0.1 mg to 1000 mg;
b1) Capsicum extract, in an amount which is adapted to provide an oral dosage of capsacinoids, comprised between 0.01 mg and 1000 mg;
b2) an Allium extract, in an amount adapted to provide a dosage of sulphur containing compounds of from 0.1 mg to 1000 mg;
b3) a Camellia extract, in an amount adapted to provide a dosage of catechin derivatives of from 0.1 mg to 3000 mg.

17. A composition according to claim 13, comprising the following amounts of the components, expressed as mg/dosage unit/die:
a) peppermint essential oil, in the amount of from 1 mg to 100 mg;
b1) Capsicum extract, in an amount adapted to provide an oral dosage of capsacinoids, comprised between 0.1 mg and 10 mg;
b2) an Allium extract, in an amount adapted to provide a dosage of sulphur containing compounds of from 1 mg to 100 mg;
b3) a Camellia extract, in an amount adapted to provide a dosage of catechin derivatives of 20 mg to 500 mg.

18. A composition according to claim 13, further comprising one or more additional components having a complementary action selected from the group consisting of:
antioxidants, particularly selected from vitamins A, C and E, SOD, CoQ10, GSH, cysteine and compounds derived from grape, blackberry, pomegranate, G. biloba and/or silimarine;
soothing agents/hypnotics/antidepressants/adaptogens, preferably of a natural origin, particularly selected from DMG, betaine, meteonine donors, Kawa-kawa, hypericus, Valerian, Passiflora, Camomile, Escolthia, Rhodiola, Ginseng, Euleterococcus and mixtures thereof;
NO donors, particularly selected from L-arginine, L-citrulline, L-ornitine;
active principles on the energetic and/or fat metabolism, preferably selected from carnitine and polycosanols.

19. A composition according to claim 13, in a form suitable for oral or sublingual administration.

20. A composition according to claim 13, in the form of hard or soft gelatine capsules, tablets, tablets or solid forms with a sustained release, with mono- or multi-layers, chewable or effervescent tablets, granular or powdery sachet, chewing-gum, oral sprays.

21. A composition according to claim 13, in the form of a unit dosage for food supplement, medical device or drug.

22. A method for treating or preventing overweight and obesity in a human subject comprising orally administering to said subject a composition according to claim 13.

23. A method for treating or preventing a condition selected from the group consisting of metabolic syndrome, hypercholesterolaemia, hypertriglyceridemia, diabetes and hypertension comprising orally administering to a human subject suffering from said condition a composition according to claim 13.
